(19) United States
(12) Patent Application Publication
(10) Pub. No.: US 2008/0021317 A1
(43) Pub. Date: Jan. 24, 2008

(54) Ultrasound medical imaging with robotic assistance for volume imaging

(75) Inventor: Thilaka Sumanaweera, Los Altos, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/492,284

(22) Filed: Jul. 24, 2006

(51) Int. Cl. A61B 8/00

(52) U.S. Cl. 600/437

(57) ABSTRACT

A robotic mechanism positions a volume scanning transducer at multiple acoustic windows on a patient. Ultrasound data is acquired from the windows and combined into a wide field-of-view. The robotic mechanism operates without user contact, such as for an automated full or partial torso scan of a patient. Alternatively, the robotic mechanism provides force to reduce strain on a sonographer.

### BACKGROUND

The present embodiments relate to ultrasound imaging. In particular, a robot assists with ultrasound imaging.

A sonographer holds a transducer for ultrasound imaging. Holding the transducer has several drawbacks. Since the transducer has only a limited field of view, the sonographer spends a lot of time trying to find the area of interest. Once the area of interest is found, only the area of interest is scanned. If there is an additional area to be investigated, the patient typically has a separate appointment for additional scanning. In contrast, computed tomography and magnetic resonance imaging use a gantry to slide the patient in and out of the scanner, acquiring data from a large area. All the data needed for the physician to diagnose the disease may be acquired during a single session.

Image quality of ultrasound depends on the sonographer and how much pressure is applied by the sonographer to the transducer against the patient. Scanning by a sonographer is expensive and prone to variability and human error. Constant application of pressure to the transducer may cause discomfort and injuries for the sonographer's fingers, wrists elbows, shoulders and neck. Scanning by a sonographer may be time consuming.

### BRIEF SUMMARY

By way of introduction, the preferred embodiments described below include methods, systems and computer readable media for ultrasound imaging with robotic assistance. A robotic mechanism positions a volume scanning transducer at multiple acoustic windows on a patient. Ultrasound data is acquired from the windows and combined into a wide field-of-view. The robotic mechanism operates without user contact, such as for an automated full or partial torso scan of a patient. Alternatively, the robotic mechanism provides force to reduce strain on a sonographer.

In a first aspect, an ultrasound system is provided for medical imaging. A robotic mechanism holds a transducer operable to scan a three-dimensional volume. The robotic mechanism includes at least one actuator operable to move the robotic mechanism in at least one degree-of-freedom. A processor is operable to receive ultrasound data representing first and second volumes acquired with the transducer held by the robotic mechanism at first and second acoustic windows, respectively, on a body. The processor is operable to combine the ultrasound data for a wide field-of-view representing at least the first and second volumes.

In a second aspect, a method is provided for medical imaging with a robotic mechanism. A robotic mechanism positions a transducer at a first position on a body. A first volume scan is performed with the transducer at the first position. The robotic mechanism positions the transducer at a second position on the body. A second volume scan is performed with the transducer at the second position. A wide field-of-view is generated from ultrasound data from the first volume scan and the second volume scan.

In a third aspect, a computer readable storage medium has stored therein data representing instructions executable by a programmed processor for ultrasound imaging with a robotic mechanism. The storage medium includes instructions for receiving spatial parameters defining a plurality of three-dimensional scan locations on or adjacent to a patient, positioning or moving, with the robotic mechanism, a transducer at or between the three-dimensional scan locations, and generating a representation of the patient from ultrasound data associated with the plurality of the three-dimensional scan locations.

In a fourth aspect, an ultrasound system is provided for medical imaging. A robotic mechanism is connectable with a volume scan transducer. A first sensor is operable to sense a first force applied by a user on the transducer, robotic mechanism or both the transducer and the robotic mechanism. A second sensor is operable to sense a second force applied to a patient. A processor is operable to control the robotic mechanism in response to the first force such that less net reactionary force is provided on a sonographer as a function of assisting force applied by the robotic mechanism while maintaining the second force below a threshold amount.

In a fifth aspect, a method is provided for medical imaging with assistance from a robotic mechanism. A first pressure applied towards a patient by a user is sensed. A second pressure applied by a transducer on the patient is sensed. The robotic mechanism applies force in response to the first pressure. The second pressure is a function of the force and the first pressure.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of an ultrasound system with a robotic mechanism;

FIG. 2 is a perspective view of one embodiment of a robotic mechanism for ultrasound imaging;

FIG. 3 is a is a graphical representation of one embodiment of a ultrasound system with a robotic mechanism in a flexible vessel;

FIG. 4 is a graphical representation of an embodiment of a shell for holding the flexible vessel of FIG. 3;

FIG. 5 shows different embodiments of shapes of the flexible vessel of FIG. 3;

FIG. 6 is a graphical representation of one embodiment of use of the ultrasound system with the robotic mechanism of FIG. 1 by a sonographer;

FIG. 7 is a graphical representation of the forces in one embodiment of the usage of FIG. 6; and

FIG. 8 is a graphical representation of one embodiment of use of the ultrasound system with the robotic mechanism of FIG. 1 without a sonographer.

### DETAILED DESCRIPTION OF THE DRAWINGS AND PRESENTLY PREFERRED EMBODIMENTS

A robotic mechanism assists with ultrasound imaging. The robotic mechanism connects with a volume transducer, such as a wobbler or multi-dimensional array. In one form of assistance, the robotic mechanism repositions the transducer to a plurality of locations for a wide-region or body type (full, torso or abdomen) scan. The volume data from each position is combined into a data set for analysis. In another form of assistance, the sonographer controls placement of the transducer by hand, but the robotic mechanism applies force in a direction indicated by the user. The user may have less strain due to the assistance by the robotic mechanism.

One or more ultrasonic probes connect with one or more robotic manipulator arms, force sensors and position sensors. A robotic manipulator arm is a mechanical device containing a series of links connected by active joints. Each joint may have a motor and a sensor sensing the angle or displacement of the joint. Each joint may have a force sensor. The probe or probes may have a pressure or force sensor.

An ultrasound system connects with the probe for acquiring B-mode, Color Doppler or Spectral Doppler information. A personal computer or other processor, such as a processor in the ultrasound system, connects with the robotic manipulator arm for controlling the arm. Separate control hardware for the robotic manipulator control may be used. The personal computer or ultrasound system includes user interface software and hardware for controlling the robotic manipulator arm. The personal computer or the ultrasound system implements computer assisted diagnosis software for generating a wide field-of-view or for analyzing ultrasound data acquired by the ultrasound system.

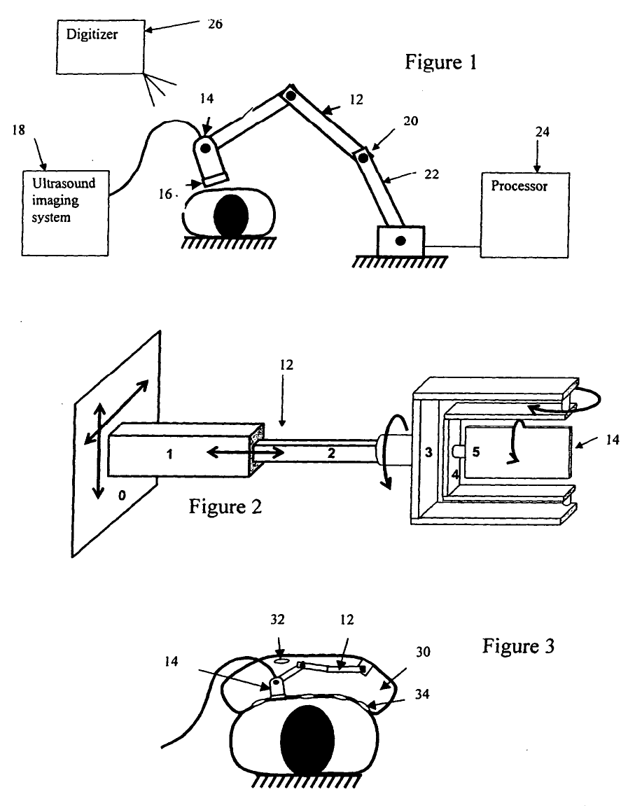

FIG. 1 shows one embodiment of an ultrasound system with a robotic mechanism 12 for medical imaging. The ultrasound system includes the robotic mechanism 12, a transducer 14, a force sensor 16, an ultrasound imaging system 18, a processor 24 and a digitizer 26. Additional, different or fewer components may be provided. For example, the ultrasound system does not include the force sensor 16 and/or the digitizer 26.

The transducer 14 is a volume scan transducer or is operable to scan a three-dimensional volume. A wobbler, multi-dimensional array (e.g., two-dimensional array transducer), or other transducer may be used. A two-dimensional array of elements may have a square, rectangular or other shaped aperture. A wobbler array may have a one or multi dimensional array of elements mechanically rotated or scanned along one or more dimensions.

The transducer 14 is mounted on, held by, mechanically connects with, or is separable from the robotic mechanism 12. For example, the transducer 14 is part of a probe with a housing for hand held use or a housing shaped for connecting to or being held by the robotic mechanism 12. The robotic mechanism 12 includes a jaw, clamp, clip, latch, micro-manipulator or other component for connecting actively or passively with the transducer 14. The robotic mechanism 12 may release the transducer 14, such as for maintenance or for use without the robotic mechanism 12. As another example, the array of elements of the transducer 14 is incorporated as part of or with the robotic mechanism. The transducer 14 may be releasable, such as for maintenance, or fixed for use without being releasable.

The transducer 14 may electrically connect with the robotic mechanism, such as having coaxial cables extending into or adjacent the robotic mechanism 12. Alternatively, the cables extend from the transducer 14 without being held by, clipped to or contained within the robotic mechanism 12.

More than one transducer 14 may be connected with the robotic mechanism 12. For example, the robotic mechanism 12 connects with two or more transducers 14 which are maintained with a particular spacing from each other or may be moved relative to each other. Separate robotic mechanisms 12 may be used for different transducers 14 or groups of transducers 14.

The robotic mechanism 12 includes one or more links 22 and actuators 20 for moving joints. The robotic mechanism 12 moves with any number of degrees of freedom, such as one to seven degrees of freedom.

The links 22 are each the same or may have different configurations, shapes, sizes, lengths or types within the same robotic mechanism 12. Any now known or later developed material may be used for the links 22, such as plastic, wood, or metal. In one embodiment, one or more of the links 22 are formed from a non-rigid flexible material, such as hard rubber. The non-rigid flexible material may assist in avoiding undo or over pressure on a patient. For example, give in the link 22 reduces pressure. The non-rigid or a rigid link 22 may be formed to break or bend in response to a threshold amount of pressure. The link 22 holding the transducer 14 and/or a link spaced from the transducer 14 may be non-rigid or yield to excessive pressure.

The links 22 connect at joints. The joints are rotatable, bendable, twistable or otherwise moveable around an axis or away from an axis of one of the links 22. Each joint may have one or more degrees of freedom.

The actuators 20 are electromagnetic, pneumatic, hydraulic or combinations thereof. One or more actuators 20 connect between the links 22 or with a joint. The actuators 20 are positioned at the joints, on links 22 or spaced from the links 22. The actuators 20 move the robotic mechanism 12 in at least one, two or more degrees-of-freedom. For example, the actuators move one link 22 relative to another link 22 by rotation, flexing, bending or other motion. The combination of actuators 20 and links 22 may allow for various positions of the robotic mechanism 12, such as seven degrees of freedom for bending or positioning around an obstacle. The actuators 20 move the transducer 14 to positions adjacent a patient's body. In one embodiment using two or more transducers 14, the actuators 20 position the transducers 14 adjacent the same body in a known or planned spatial relationship.

The actuators 20, with or without additional sensing, may allow for back driving of the robotic mechanism. For example, the actuators 20 allow a person to move the robotic mechanism with minimal force away from a patient. Alternatively or additionally, the actuators 20 and/or links 22 include one or more locks or are resistant to movement from external sources.

The robotic mechanism 12 includes one or more sensors, such as position, force, pressure, displacement, or other types of sensors. In one embodiment, a position sensor connects to the transducer 14, the robotic mechanism 12 or both the transducer 14 and the robotic mechanism 12. For example, an ultrasound, magnetic, optical or other position sensor indicates the position of the transducer 14 relative to a room, patient or robotic mechanism. As another example, angle or rotation sensors, such as optical or resistive encoders, determine a position of the transducer 14 from the relative positions of the different links 22 and/or a base of the robotic mechanism 12 based on or relative to a known position of the base.

Another sensor may be the force sensor 16. The force sensor 16 is piezoelectric, capacitive, strain gauge or other sensor operable to indicate pressure. The force sensor 16 connects to the transducer 14, the robotic mechanism 12 or both the transducer 14 and the robotic mechanism 12. For example, the force sensor 16 is adjacent or over an acoustic window of the transducer 14 for sensing pressure applied to a patient. As another example, the force sensor 16 is one or more sensors for determining pressure or strain at one or more locations on the robotic mechanism. The pressure measurement from the robotic mechanism 12 may be used to determine a pressure applied to the patient.

Another sensor may be another force sensor positioned on the transducer 14, the robotic mechanism 12 or both to sense user applied pressure. The sensor is positioned to determine an amount and/or direction of pressure applied by a sonographer. The robotic mechanism 12 may respond to sonographer-applied pressure to increase or decrease pressure applied to the patient or to assist in moving the robotic mechanism 12 with the actuators 20. In combination with the force sensor 16, the force applied to the patient by the sonographer and the robotic mechanism 12 is limited, but the force applied by the sonographer may be less than the force applied to the patient.

The robotic mechanism 12 with or without use of the sensors may be used for strain, elastography and/or palpation imaging. For example, the robotic mechanism 12 vibrates the transducer 14 at a controllable palpation frequency or using a palpation pulse. As another example, images associated with different amounts of pressure applied to the patient by the transducer 14 are acquired for strain or elastography determinations.

The robotic mechanism 12 avoids uncontrollable movement during mechanical or electrical failure, such as a mechanical fuse assuring safe operation. The actuators 20 may operate at any speed, but only allow slow motion in one embodiment. For example, high gear reduction ratios, low power drives, and/or stepper motors prevent movements that may concern patients. Damping motion may limit dynamic performance. A dead-man's switch may be used to minimize stop time where the sonographer releases the switch. The switch may be a foot peddle, hand switch or other device. Unobtrusive designs may be used, such as covering the robotic mechanism 12 with soft or gently curving housings.

The robotic mechanism 12 includes a gel dispenser and a suction spout. Tubes provide gel from a reservoir on or off the robotic mechanism 12. A pump forces gel from the gel dispenser onto the patient where the transducer 14 is to be positioned. The gel dispenser is adjacent the transducer 14 or is on a separate link 22. The suction spout connects with a vacuum source for removing gel from the patient. The suction spout is adjacent the transducer 14 or is on a separate link 22. Gel dispensing and/or suction or cleaning may be performed manually.

FIG. 2 shows another embodiment of the robotic mechanism 12. Six degrees of freedom are provided where expected motion for scanning a patient is mostly linear. Linear motion between links 0-1 and 1-2 provides translation. The transducer 14 is able to rock, roll and pitch around the transducer's lens or end of the transducer 14. A force sensor may be provided, such as a force sensor between links 4-5. Other or additional locations are possible. Other robotic mechanisms may be provided with fewer or more links, actuators, and/or joints.

The robotic mechanism 12 extends from a table, cart, wall, ceiling or other location. The base may be fixed or mounted, but alternatively is releasable or merely rests due to gravity on an object. The robotic mechanism 12 extends from the mount to the patient for scanning with the transducer 14.

In an alternative embodiment, FIG. 3 shows the robotic mechanism 12 encapsulated, at least partly, inside a fluid-filled flexible bag 30. The robot mechanism 12 is encapsulated completely or partially inside the fluid-filled flexible bag 30. The bag 30 is an acoustically transparent pillow of Urethane or other material that conforms, at least in part, to the patient's body. The fluid is de-gassed water doped with PED (polyethelene glycol) or other liquid.

An acoustic coupling gel-pad, such as AQUAFLEX available from Parker Labs, is molded into a portion of the bag 30 or positioned between the patient and the bag 30. This pad is placed between the patient and the pillow for good acoustic coupling. Alternatively, gel is manually positioned on the patient prior to placing the bag 30 on the patient. The robot mechanism 12 made in part or in full using flexible material holds the transducer 14 and presses the transducer 14 against the inside of the bag 30, making contact with the inside skin of the bag 30. Alternatively, the transducer 14 does not touch the bag 30, such as being maintained a fixed distance from the inside skin of the bag 30.

The robotic mechanism 12 has 1 or more, such as 6, degrees of freedom. For example, the robotic mechanism 12 includes rails guiding the transducer 14 inside the bag 30, or any other mechanism capable of guiding the transducer 14 in three dimensions with arbitrary orientations. A force sensor or sensors ensure application of the correct pressure against the inside walls of the bag 30. The transducer position and orientation in 3D space is determined either using the robotic mechanism's joint angle or locations or using independent position sensors, such as magnetic, laser-based, laser range finder-based, camera-based, LED-based or using any other type of position sensors.

Flexible cable, such as a ribbon of coaxial cables, connects the transducer 14 to the ultrasound imaging system 18 through the bag 30. Force or pressure sensors 34 inside the bag 30, inside the bag wall, between the bag 30 and any gel-pad, inside any gel-pad, between the patient and the gel-pad, or any combination thereof, monitor the pressure against the patient. Sufficient pressure against the patient more likely provides good acoustic coupling. A pressure sensor 32 inside the fluid bag 30 monitors the pressure of the fluid to warn of excessive or insufficient fluid pressure.

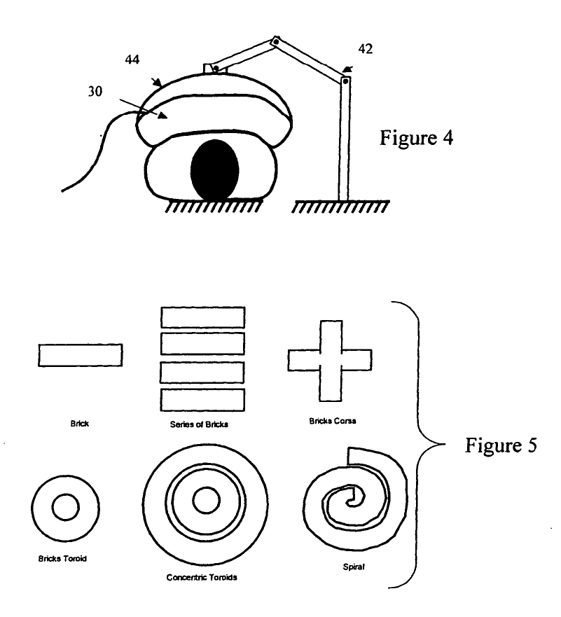

FIG. 4 shows an external shell 44 holding the bag 30 to make sure that undue pressure is not applied on the patient due to gravity and/or to provide more stable operation of the robotic mechanism 12. The shell 44 is flexible or inflexible material attached to the bag 30. The shell 44 is mounted on a passive articulated arm 42. The arm 42 may be robotic in other embodiments.

FIG. 5 shows different embodiments for the shape of the bag 30 from a top view. A flexible rectangular brick, a tube, a series of bricks, a brick cross, a brick toroid, concentric toroids, a spiral brick, a brick shaped into a helix or any other shape may be used. The helix, brick, toroid or other structure may allow for one robotic mechanism 12 to transition along rails for scanning at a plurality of acoustic windows. The concentric toroids, series of bricks or other structures may use a plurality of robotic mechanisms 12 and transducers 14 in separate bags 30.

Referring to FIG. 1, the ultrasound imaging system 18 is a B-mode, Doppler, flow or other imaging system. A beamformer, detector, scan converter and display generate ultrasound images using the transducer 14. A three-dimensional processor receives ultrasound data for three-dimensional imaging or conversion to a three-dimensional grid. Projection, surface or other types of rendering may be performed.

In one embodiment, the processor 24 is part of the ultrasound imaging system 18. Alternatively, the processor 24 is a separate device for controlling the robotic mechanism 12 and/or generating three-dimensional images or data.

The digitizer 26 is a laser range finder, scanner, optical sensor or other device operable to determine the geometry of at least a portion of the patient. For example, a grid is transmitted onto the patient. A charge-coupled device or other optical device images the grid as projected onto the patient. Deviations of the projected grid, such as curved lines, indicate the depth or surface of the patient. Range finding may be used to determine the distance to the surface. Other now known or later developed devices for determining a geometry and/or location of the surface of the patient may be used.

The processor 24 controls the robotic mechanism 12 at least in part based on the geometry of the surface. The processor 24 is a general processor, digital signal processor, application specific integrated circuit, field programmable gate array, analog device, digital device, combinations thereof or other now known or later developed controller. The processor 24 is separate from or part of the digitizer 26 and/or the ultrasound imaging system 18. In one embodiment, the processor 24 is a personal computer or general processing board. In another embodiment, the processor 24 includes a plurality of devices for parallel or sequential processing. For example, the processor 24 includes a general processor or programmable device and a separate hardware interface with the robotic mechanism 12. The separate interface may allow any device operable to output a standard set of codes to control the robotic mechanism 12. The separate interface may also allow for redundant pressure sensing or safety controls.

The processor 24 determines locations for scanning on the patient with the volume transducer 14. The sonographer or user indicates locations, such as selecting points on a displayed image of the patient or controlling with a joystick. The locations may be manually programmed. The locations may be set by the user placing the transducer 14 in multiple locations, and the processor 24 recording the positions.

Using the digitizer 26, the processor 24 may identify features of the patient for automatic determination of scanning locations. The output geometry of the surface of the patient from the digitizer 26 identifies the features or locations of different portions of the patient. Acoustic windows relative to the features of the patient are then determined by the processor 24 without further user input. The force sensor 16 may be used to determine features with or without also using the digitizer 26. For example, the pressure due to various bones being contacted by the force sensor 16 may allow mapping of the geometry of the patient.

A plurality of acoustic windows is determined from the geometry of the surface of the patient. The acoustic windows correspond to tissue locations with a partial or complete acoustic view of the patient's interior. The acoustic windows may be associated with holes, such as between ribs, or with generally open locations, such as a region of the abdomen. The acoustic windows are determined automatically, such as selecting a sequence of acoustic windows to scan an extended volume of the patient. In one embodiment, the user may select one or more starting, ending or intermediate locations for scanning, and the processor 24 determines other acoustic windows. Preset acoustic window location patterns or operator-selected patterns may be used.

Once a map for scanning is provided, the processor 24 controls the robotic mechanism 12 to position the transducer 14 at the different or sequence of acoustic windows. The processor 24 controls the actuators without user contact with the robotic mechanism 12. The robotic mechanism 12 scans the patient automatically by changing the location and orientation of the transducer 14 and the pressure applied by the transducer 14 on the patient. The robotic mechanism 12 effectively acts as a gantry, such as a gantry of a CT or MRI system. The sonographer is absent from scanning, so does not contact the transducer 14 or the robotic mechanism 12 during scanning or positioning for scanning at different acoustic windows. The trajectory of scanning is preset or computed on the fly, such as comparing the acquired images with stored data (e.g., a catalog of ultrasonic images, atlases or other descriptions). The acoustic windows may be repositioned to assure alignment of the scans and/or scanning desired internal structures.

The processor 24 receives ultrasound data from the transducer 14 at each acoustic window. The processor 24 may have a separate component for receiving ultrasound data, such as the ultrasound imaging system 18 being part of the processor 24. Alternatively, the processor 24 receives data output by the ultrasound imaging system 18 or the transducer 14.

The transducer 14 is used to scan a volume at each acoustic window. A pattern of scan lines with both elevation and azimuth distributions is used. For example, data from a plurality of elevationally spaced planes is acquired. Any three-dimensional or volume scan pattern may be used. The received ultrasound data represents different volumes acquired with the transducer 14 held by the robotic mechanism 12 at the different acoustic windows. The received ultrasound data is in a polar, Cartesian or other format. For example, the data is interpolated to a regularly spaced three-dimensional grid. As another example, the data is entirely in a polar coordinate format associated with the scan pattern. In another example, the relative spacing of planes is in polar coordinate format but the data for each plane is scan converted to a Cartesian format.

The processor 24 combines the sets of ultrasound data for a wide field-of-view. Each set of data corresponds to a different scanned volume. The acoustic windows are selected for scanning overlapping or adjacent volumes. Using position sensors or the relative position of the robotic mechanism 12 or transducer 14, the relative position of the scanned volumes is determined or known. Alternatively or additionally, data correlation may be used to determine the relative position of the volumes, such as using a search pattern with minimum sum of absolute differences or other correlation. Other techniques, such as those used in U.S. Pat. No. 5,965,418, the disclosure of which is incorporated herein by reference, may also be used.

The ultrasound data representing the volumes may be combined to represent a larger volume or wide field-of-view. The combination may be by averaging or weighted combination of data representing the same or similar locations from different data sets. One of a plurality of values representing a same or similar location may be selected to provide combination of the sets of data. Where overlap does not occur, the combination may include forming a larger volume and positioning the ultrasound data conceptually within the larger volume, such as aligning the data sets as a function of relative position.

In one embodiment, any of the systems, methods, or computer readable media disclosure in U.S. Patent Application Publication Nos. 2005/0033173 and ______ (application Ser. No. 11/415,587, filed May 1, 2006), the disclosures of which are incorporated herein by reference, may be used. Other combinations may be used, such as disclosed in U.S. Pat. Nos. 5,876,342, 5,575,286, 5,582,173, 5,782,766, 5,910,114, 5,655,535, 5,899,861, 6,059,727, 6,014,473, 6,171,248, 6,360,027, 6,364,835, 6,554,770, 6,641,536 and 6,872,181, the disclosures of which are incorporated herein by reference. Processes taught in the above referenced patents for two-dimensions may be extended to three-dimension processes.

Due to the pressure applied by the transducer 14 at each acoustic window, the volume represented by the ultrasound data may be warped or altered. The alteration may be acceptable without further processing. Alternatively, rigid body or non-rigid body transformations between data sets may be performed prior to or as part of the combination. For example, any of the transformations disclosed in U.S. Pat. No. 6,306,091, the disclosure of which is incorporated herein by reference, may be used.

By scanning a wide region using multiple acoustic windows, the resulting ultrasound data set or examination is similar to CT and MRI, where a gantry is used to scan a wide region of interest. With the force feedback, the articulated robotic mechanism 12 can apply a desired pressure for optimal image-quality. Other heuristic knowledge, such as relative locations of organs to be expected, can be encoded in the processor 24 for obtaining the best image-quality, quickly. The results may be operator-independent and more repeatable.

The processor 24 may extract, automatically, a subset of the ultrasound data associated with scanned structure of the body. The combination allows extraction of data from different scanned volumes. The extraction may be from data in a uniform or combined volume or from different data sets having a known spatial relationship from the combinations of volumes. The extracted data may be used to generate an image of a specific organ or other region or for calculating diagnostic information, such as borders, surfaces, textures, lengths, or flow.

With or without extraction, the processor 24 may measure, automatically, a quantity associated with structure of the body from the ultrasound data. The known spatial relationship or the combined data allows calculation of lengths, volumes or other spatial quantities extending between different volumes or within an extended volume.

The processor 24 may perform computer-aided diagnosis (CAD), such as automatically extracting a suitable subset of data from the composite data sets for the physician or to determine a disease state. Increased specificity and/or sensitivity may be provided by the consistency of scanning with the robotic mechanism 12. The needs of the computer-assisted diagnosis may be used to influence or control the scanning by the robotic mechanism 12, such as to gather more data if a decision is inconclusive using Color Doppler or Spectral Doppler.

For a specific computer assisted diagnosis example, abdominal aortic aneurysms (AAA) or patients at risk for AAA are detected. For patients with the potential for bowel gas, the patient is asked to avoid eating prior to scanning. By constantly monitoring the pressure of the transducer and velocities of the blood, a quantitative basis for the safety of the system is provided to avoid accidental rupture of the AAA during scanning. In one embodiment, the flow processes disclosed in U.S. Pat. No. 6,503,202, the disclosure of which is incorporated herein by reference, are used to detect AAA or AAA risk.

As another example, carotid artery screening is provided. People with carotid artery disease may have increased risk for stroke, myocardial infarction and death. The robotic mechanism 12 scans a patient's carotid artery automatically or from joystick control. Plaque build-up inside the artery is identified from the ultrasound information. Rupture or release of the plaque may be limited or more likely avoided by using the robotic mechanism 12.

In another example, Hepatocellular Carcinoma (HCC) screening is provided. The liver of a person is scanned. If detected early, HCC can be cured completely. Contrast agent imaging is used to determine perfusion in the liver for diagnosis of HCC.

The processor 24 includes or connects with a memory. The memory is a computer readable storage medium having stored therein data representing instructions executable by the programmed processor 24 for ultrasound imaging with a robotic mechanism. The instructions for implementing the processes, methods and/or techniques discussed above are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

In one embodiment, the instructions are for generating spatial parameters from output data of a sensor, such as the digitizer. Alternatively or additionally, the programmed processor 24 receives spatial parameters defining a plurality of three-dimensional scan locations on or adjacent to a patient, positions, with the robotic mechanism 12, the transducer 14 at the three-dimensional scan locations, and generates a representation of the patient from ultrasound data associated with the plurality of the three-dimensional scan locations.

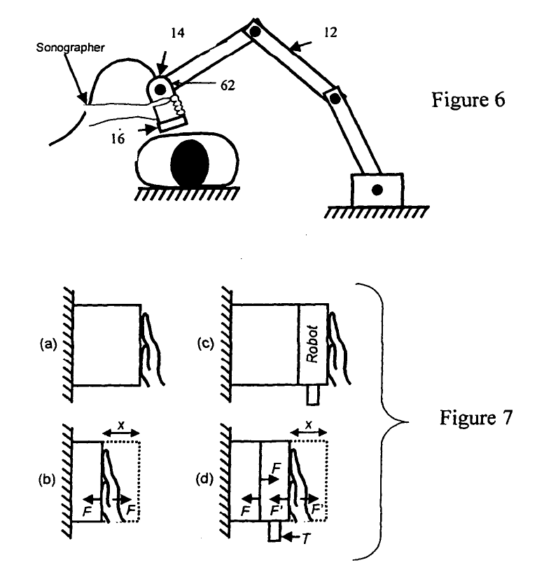

FIG. 6 shows another embodiment for ultrasound imaging with the robotic mechanism 12. The robotic mechanism 12 assists the user for scanning while the user holds the transducer 14 or a portion of the robotic mechanism 12. A force sensor 62 determines an amount or direction of force applied by the sonographer to the transducer 14 or the robotic mechanism 12. The force sensor 62 is positioned under the user's hand during use or elsewhere along the robotic mechanism 12. Another force sensor 16 determines the force applied to the patient in one embodiment. Alternatively, the assisted movement by the robotic mechanism is not towards the patient.

The robotic mechanism 12 generates part of the movement or pressure force, such as part of the force pressing the transducer 14 against the patient. The force is generated in response to the sonographer applying force in the desired direction. The processor 24 determines the pressure applied to the body as a function of output from the force sensor 16. Actuators 20 are controlled in response to both force sensors 16, 62. The robotic mechanism 12 applies some of the force against the patient or moves in response to the user applying some force. For pressing against the patient, the robotic mechanism 12 applies force such that less net reactionary force is provided on a sonographer as a function of assisting force applied by the robotic mechanism 12 while maintaining the force against the patient below a threshold amount.

For example, the sonographer images a patient using the transducer 14 mounted on the robotic mechanism 12. The robotic mechanism 12 is unobtrusive, unthreatening, lightweight and quick reactive, such as through size, shape and stiffness. The sonographer can move the transducer 14 the same way he/she currently does during scanning without any hindrance from the robotic mechanism 12. When needed, the sonographer activates a power-assist feature so that the robotic mechanism 12 assists in motion or applying pressure.

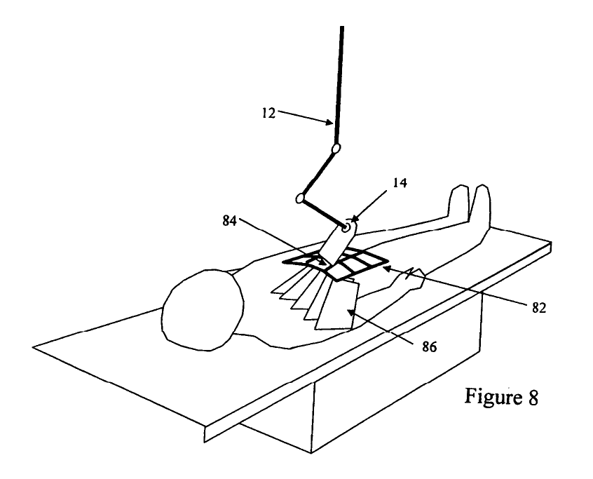

FIG. 7(a) shows pressing an elastic surface or object. If the object is compressed by a distance x and stopped, the force applied on the object and the force applied by the object on the sonographer is F=kx, where k is the stiffness. Stiffness is related to the Young's modulus of the object. FIGS. 7(c)-(d) show pressing the same object with assistance by the robotic mechanism 12. The robotic end-effector (e.g., transducer 14) is in between the sonographer and the object (e.g., the patient). If the end-effector is pressed and moved by a distance x, the force applied by the end-effector on the object and the force applied by the object on the end-effector is still F. However, the actuators 20 on the robot mechanism 12 apply a force Ton the robotic end-effector. The force applied by the sonographer on the end-effector and the force applied by the end-effector on the sonographer is F′=F−T=F(1−α) where α is the fraction of the earlier force applied by the actuators 20 of the robotic mechanism 12 so that: T=Fα. The effective stiffness of the object is k′=F′/x=k(1−α). With the robotic mechanism 12, the sonographer feels a lower force than without the robotic mechanism 12. The object's stiffness will feel more elastic. The user or the processor 24 sets a value of α to obtain the desired stiffness.

Once a region of interest is found on the abdomen, the sonographer applies the desired pressure using his/her fingers, wrist and elbows on the transducer 14. However, the actuators 20 in the joints of the robotic mechanism 12 assist the sonographer by supplying an assisting force. The sonographer feels less force to scan.

FIG. 8 represents a method for medical imaging with a robotic mechanism 12. The robotic mechanism 12 allows the sonographer to scan with the transducer 14, such as while the transducer 14 is connected with or separated from the robotic mechanism 12. Automated scanning is activated when desired. Alternatively, the robotic mechanism 12 is used only for automated scanning.

The transducer 14 is positioned with the robotic mechanism 12 at a first position on a body, such as the position 84 of a scanning grid or map 82. In one embodiment, the user positions the transducer 14 and the robotic mechanism 12 at a starting and/or other locations.

Alternatively, the robotic mechanism 12 positions the transducer 14 without control or force from the sonographer. A sensor determines the geometry of the body of a patient. A body scan map 82 is generated as a function of the geometry. The body scan map 82 includes a plurality of positions or acoustic windows 84 for scanning. The robotic mechanism is controlled automatically as a function of the body scan map 82. The transducer 14 is positioned at the different positions as a function of a body scan map 82. The positioning is performed without user applied force to the robotic mechanism.

The positions may be determined based on ultrasound data received at other positions. For example, an orientation or position of an internal organ is identified by from a prior scan. The expected location of the remainder or other portion of the organ is determined by a processor with or without additional input from a user. One or more other windows for scanning the remainder of the organ are determined.

The amount of pressure applied by the robotic mechanism 12 may be controlled. A preset is provided. Alternatively, a force sensor determines an amount of force applied by a sonographer when positioning the transducer 14 and applies the same force when not held by the sonographer.

The transducer 14 is used for a volume scan with the transducer 14 at the starting position 84. FIG. 8 shows a plurality of scan planes 86 for volume scanning while the transducer 14 is at the position 84. Any number of scan planes or other volume format may be used. For a wobbler or other transducer, the scanning of the planes may be performed in response to a trigger, such as scanning at the R-wave or other ECG trigger event.

After completing a volume scan, the robotic mechanism 12 positions or moves the transducer 14 at or to another position on the body. Another acoustic window 84 in the grid or map 82 is selected, either automatically or based on user input or control. The robotic mechanism 12 moves the transducer 14 to the next acoustic window 84 with or without assistance from the sonographer. Another volume scan is performed using the transducer 14 at the next acoustic window 84. By repeating the positioning and volume scanning with the robotic mechanism 12, automatic sweeps are provided from a starting point.

The map or grid 82 shows nine acoustic windows 84. Greater or fewer windows may be used in a regular or irregular pattern. Axial, sagittal, coronal and/or other sweeping patterns may be used. While imaging the abdomen is shown, other portions of the patient may be imaged.

Once two or more volumes are scanned, a wide field-of-view is generated from the ultrasound data of the scanned volumes. The volumes spatial position is registered or aligned to generate a composite 3D volume or 4D volumes (e.g., a sequence of composite 3D volumes). Rigid-body and/or non-rigid body registrations may be used. Any spatial compounding may be used for overlapping positions. Combining data from different scans may lower speckle variance, compensate for signal loss, and/or reduce artifacts.

The combined volume is used to generate an image, determine a quantity or for computer assisted diagnosis. For imaging, any rendering of a three-dimensional representation may be used. A multiplanar reconstruction from data of two or more volumes may be generated. The user or the processor selects the planes for reconstruction.

The robotic mechanism scans the target automatically using B-mode, Color Doppler, Spectral Doppler, and/or other modes. By scanning in multiple modes, different types of data or information are available for later diagnosis based on an earlier scan. A CAD system may analyze the data and present a score based on the severity of any disease for an initial or second diagnosis. If the score is high or diagnosis is confirmed by a sonographer or physician, the patient is scanned pursuant to traditional ultrasound approach, such as scanning for a particular concern with guidance or control by a sonographer. If the score is low or after a negative diagnosis is confirmed, no further action is needed. By avoiding a sonographer for the initial scan, costs may be reduced (e.g., only people who score high go to the hospital to be scanned by sonographers), and the examination or diagnosis may be performed more quickly. More widespread screening may be available, even for persons in lower risk categories, such screening women who have smoked for AAA.

In another embodiment, the robotic mechanism provides assistance to the sonographer while the sonographer controls or positions the transducer. A map 82 is or is not used. The robotic mechanism provides pressure or strain relief to the sonographer. A pressure applied towards a patient by a sonographer is sensed. The pressure is applied while the transducer is in contact with the patient. Another pressure being applied to the patient by the transducer is sensed. The robotic mechanism applies force in response to the pressure applied by the sonographer. The pressure applied to the patient is a combination of the pressure applied by the robotic mechanism and the pressure applied by the sonographer. The desired pressure for scanning is applied without the sonographer having to apply the full pressure. The robotic mechanism may be adjusted or controlled to increase or decrease the amount of pressure applied to the patient and/or amount of pressure needed to be applied by the sonographer.

In other embodiments, pressure or force applied in any direction, including not towards the patient, is sensed. The robotic mechanism assists in moving the robotic mechanism, reducing the force needed to be applied by the sonographer to move the robotic mechanism.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. An ultrasound system for medical imaging, the ultrasound system comprising:
a transducer operable to scan a three-dimensional volume;
a robotic mechanism with at least one actuator operable to move the robotic mechanism in at least one degree-of-freedom; and
a processor operable to receive ultrasound data representing first and second volumes acquired with the transducer held by the robotic mechanism at first and second acoustic windows, respectively, on a body, and the processor operable to combine the ultrasound data for a wide field-of-view representing at least the first and second volumes.

2. The ultrasound system of claim 1 wherein the robotic mechanism comprises one or more sensors.

3. The ultrasound system of claim 1 wherein the transducer comprises a wobbler or a two-dimensional array of elements.

4. The ultrasound system of claim 1 wherein the processor is operable to extract automatically a subset of the ultrasound data associated with scanned structure of the body.

5. The ultrasound system of claim 1 wherein the processor is operable to a measure automatically a quantity associated with structure of the body from the ultrasound data.

6. The ultrasound system of claim 1 further comprising a second transducer connected with the robotic mechanism, wherein the robotic mechanism is operable to position the transducer and the second transducer adjacent the body in a known spatial relationship.

7. The ultrasound system of claim 1 wherein the robotic mechanism comprises at least one link of non-rigid flexible material.

8. The ultrasound system of claim 1 further comprising a position sensor connected to the transducer, the robotic mechanism or both the transducer and the robotic mechanism.

9. The ultrasound system of claim 1 further comprising a force sensor connected to the transducer, the robotic mechanism or both the transducer and the robotic mechanism, the processor operable to determine a pressure applied to the body as a function of output from the force sensor.

10. The ultrasound system of claim 1 wherein the robotic mechanism is encapsulated, at least partly, inside a fluid-filled flexible bag.

11. The ultrasound system of claim 1 wherein the at least one actuator comprises an electromagnetic actuator, pneumatic actuator, hydraulic actuator or combinations thereof.

12. The ultrasound system of claim 1 wherein the processor is operable to position the transducer at the first and second acoustic windows by control of the actuator without user contact with the robotic mechanism.

13. The ultrasound system of claim 1 further comprising a geometry digitizer operable to determine a geometry of a surface of the body, the processor operable to determine the first and second acoustic windows as a function of output of the geometry of the surface.

14. The ultrasound system of claim 1 further comprising a sensor operable to sense user applied pressure to the robotic mechanism, the transducer or both the robotic mechanism and the transducer;
wherein the processor is operable to control the actuator as a function of output from the sensor.

15. The ultrasound system of claim 1 wherein the robotic mechanism is operable to apply and remove ultrasonic gel on the body.

16. The ultrasound system of claim 1 wherein the processor is operable to combine the ultrasound data for the wide field-of-view representing at least the first and second volumes as a function of positions of the robotic mechanism while holding the transducer at the first and second acoustic windows.

17. A method for medical imaging with a robotic mechanism, the method comprising:
positioning a transducer with a robotic mechanism at a first position on a body;
performing a first volume scan with the transducer at the first position;
positioning the transducer with the robotic mechanism at a second position on the body;
performing a second volume scan with the transducer at the second position; and
generating a wide field-of-view from ultrasound data from the first volume scan and the second volume scan.

18. The method of claim 17 wherein positioning comprises positioning at the first and second positions as a function of a body scan map.

19. The method of claim 17 further comprising:
determining, with a sensor, a geometry of the body;
generating a body scan map as a function of the geometry, the body scan map including the first and second positions; and
controlling the robotic mechanism as a function of the body scan map;
wherein the positioning is performed without user applied force to the robotic mechanism.

20. In a computer readable storage medium having stored therein data representing instructions executable by a programmed processor for ultrasound imaging with a robotic mechanism, the storage medium comprising instructions for:
receiving spatial parameters defining a plurality of three-dimensional scan locations on or adjacent to a patient;
moving, with the robotic mechanism, a transducer between the three-dimensional scan locations; and
generating a representation of the patient from ultrasound data associated with the plurality of the three-dimensional scan locations.

21. The instructions of claim 20 further comprising:
generating the spatial parameters from output data from a sensor.

22. An ultrasound system for medical imaging, the ultrasound system comprising:
a volume scan transducer;
a robotic mechanism connectable with the volume scan transducer;
a first sensor operable to sense a first force applied by a user on the transducer, robotic mechanism or both the transducer and the robotic mechanism;
a second sensor operable to sense a second force applied to a patient; and
a processor operable to control the robotic mechanism in response to the first force such that less net reactionary force is provided on a sonographer as a function of assisting force applied by the robotic mechanism while maintaining the second force below a threshold amount.

23. The ultrasound system of claim 22 wherein the robotic mechanism comprises at least one actuator operable to generate a first portion of the second force in response to the sonographer applying the first force, at least part of the first force comprising the second force.

24. The ultrasound system of claim 22 wherein the volume scan transducer comprises a wobbler or a two-dimensional array;
wherein the robotic mechanism has at least two-degrees of freedom;
further comprising a position sensor operable to determine a position of the transducer.

25. A method for medical imaging with assistance from a robotic mechanism, the method comprising:
sensing first pressure applied towards a patient by a user;
sensing second pressure applied by a transducer on the patient; and
applying force with the robotic mechanism in response to the first pressure, the second pressure being a function of the force and the first pressure.
